(19) United States
(12) Patent Application Publication
(10) Pub. No.: US 2007/0054276 A1
(43) Pub. Date: Mar. 8, 2007

(54) Polynucleotide analysis and methods of using nanopores

(76) Inventor: Jeffrey R. Sampson, San Francisco, CA (US)

Correspondence Address: AGILENT TECHNOLOGIES, INC.;Intellectual Property Administration, Legal Department, DL429, P.O. Box 7599, Loveland, CO, 80537-0599, US

(21) Appl. No.: 10/916,931

(22) Filed: Aug. 12, 2004

(51) Int. Cl. C12Q 1/68

(52) U.S. Cl. 435/6; 977/924

(57) ABSTRACT

Polynucleotide analysis systems and methods of nanopore analysis are provided.

### BACKGROUND

Determining the nucleotide sequence of DNA and RNA in a rapid manner is a major goal of researchers in biotechnology, especially for projects seeking to obtain the sequence of entire genomes of organisms. In addition, rapidly determining the sequence of a nucleic acid molecule is important for identifying genetic mutations and polymorphisms (e.g., single nucleotide polymorphisms (SNP)) in individuals and populations of individuals.

The use of SNPs as genetic markers for locating genes associated with a specific disease is rapidly becoming standard practice within the pharmaceutical and biomedical research industries (Rotherberg, B. E. G., Nature Biotechnology 19, 209, (2001) & Sachidanandam R., et al., Nature 409, 928 (2001)). It is however becoming increasing clear from recent genetic analyses, that because of the variability of linkage disequilibrium along the human genome, there is not likely to be a simple positional relationship between SNPs and defined disease genes (Stephens J. C. et al., Science 293, 489 (2001) & Reich D. E., et al., Nature 411, 199 (2001)). Rather, the genetic evidence indicates that the necessary correlation between SNPs and disease genes will come from a detailed understanding the genetic haplotype, which is defined by the allele identity for clusters of SNPs located along the same physical chromosome.

In principle, the number of potential haplotypes (allele combinations) within a defined chromosomal segment can be quite large. For example, if one assumes an average density of one SNP per 1,000 base-pairs, and that all SNPs are biallelic, than in any given 100 Kb chromosomal fragment there is a possible 2¹⁰⁰ combinations, or 1×10³⁰. Importantly however, the number of actual haplotypes within the entire human population is much smaller (Stephens J. C. et al., Science 293, 489 (2001)). This again is a result of the genetic disequilibrium within the human genome, which is due to the fact that the extant human genetic population is relatively young. As such, there is a growing need to determine the range of haplotype identities that exist within the current human population and to establish their relationship to defined diseases. Once these correlations are made and the pharmacogenomic approach to medicine becomes established, determining the haplotype of individual patients will become a necessary part of standard medical practice.

Nanopore technology is one method of rapidly detecting nucleic acid molecules. The concept of nanopore sequencing is based on the property of physically sensing the individual nucleotides (or physical changes in the environment of the nucleotides (i.e., electric current)) within an individual polynucleotide (e.g., DNA and RNA) as it traverses through a nanopore aperture. The use of membrane channels to characterize polynucleotides as the molecules pass through a small ion channel has been studied by Kasianowicz et al. (Proc. Natl. Acad. Sci. USA. 93:13770-3, 1996, incorporate herein by reference) by using an electric field to force single-stranded RNA and DNA molecules through a 2.6 nanometer diameter nanopore aperture (i.e., ion channel) in a lipid bilayer membrane. The diameter of the nanopore aperture permitted only a single strand of a polynucleotide to traverse the nanopore aperture at any given time. As the polynucleotide traversed the nanopore aperture, the polynucleotide partially blocked the nanopore aperture, resulting in a transient decrease of ionic current. Since the length of the decrease in current is directly proportional to the length of the polynucleotide, Kasianowicz et al. were able to determine experimentally lengths of polynucleotides by measuring changes in the ionic current.

In this regard, the Oligonucleotide Encoded Hybridization Assay (OEHA) can be an effective method for both identifying specific DNA targets within a complex sample mixture and determining the allele identity of one or more SNPs on that target within the sample mixture (e.g., U.S. Patent Application 20030104428). In this method, short oligonucleotides (i.e., between 15 and 25 nucleotides) are designed to specifically hybridize along a targeted polynucleotide sequence in such a way as to generate a defined ionic current pattern as the alternating stretches of single and double-stranded regions of the target molecules traverse the nanopore. In this way, any given target's identity within the mixture can be established from the ion current pattern resulting from the duplexes generated by the “encoding oligonucleotides” (EO). The allele identity of a given SNP on the target is then determined by whether or not an “allele-discriminating oligonucleotide” (ADO) directed to hybridize to one of the two potential alleles is indeed hybridized to target.

Because the nanopore measurement process is designed to measure individual target molecules, it is critical that the analysis obtained for a single, or small number of defined target molecules to in fact be representative of the state of all target molecules of a single identity. For this attribute to hold true, the method for both encoding the target molecules within the sample mixture and identifying specific alleles along the encoded molecule must result in overall molecular states (single stranded vs duplex) that are as binary as possible. Otherwise, a large distribution of encoded molecular states will require the measurement of a large number of molecules of a given identity in order to establish a distribution upon which the target identification and/or allele identity determination is made. Clearly, this situation will greatly increase the sample analysis time and hence decrease the value of the method.

The ability to create highly homogenous molecular states within a sample mixture will be dictated by the specificity of the EOs and ADOs for their intended target sequences within the sample mixture. The hybridization specificity of the EOs and the ADOs is a function of the thermodynamic and kinetic properties of the resulting EO/target and ADO/target duplexes. These properties will be both sequence and solution-condition dependent. The stability of any given EO/target or ADO/target duplex and related mismatches can be calculated for a defined set of solution conditions using empirically determined standard ΔG^{o}, ΔH^{o} and ΔS^{o} values for nearest neighbor sequences (SantaLucia, J., Proc. Natl. Acad. Sci. USA, 95, 1460 (1998), Allawi, H. T. & SantaLucia J., Biochemistry 37, 9435 (1998), Allawi, H. T. & SantaLucia, J. Nucleic Acids Res. 26, 2694 (1998), Peyret, et al., Biochemistry 38, 3468 (1999), Allawi, H. T. & SantaLucia, J. Biochemistry 37, 2170 (1998) & Allawi, H. T. & SantaLucia, J. Biochemistry 36, 105810 (1997)).

By way of example, the calculated ΔH^{o}, ΔS^{o} and ΔG^{o} values (@ 1 M NaCl, ˜1.0 nM each strand, 37° C.) for a 19 mer DNA/DNA duplex and its related single mismatches (G/A, G/G, G/T) as well as the multiple quadruple and quintuple internal mismatched duplexes are calculated in Table 1 below.

TABLE 1
Calculated ΔH°, ΔS° and ΔG° Values

| | | | |
|---|---|---|---|
| | Delta H | Delta S | Delta G |
| Duplex Sequence (mismatch indicated as N/N) | (cal/mol) | (cal/K mol) | (cal/mol) |

| | | | | |
|---|---|---|---|---|
| Central Base Pair | | | | |
| Mismatches | | | | |
| 19 mer | G G A C A T A C C G A G T G A A T C G | −149,300 | −407 | −23,285 |
| 19 mer: G/A | G G A C A T A C C G/A A G T G A A T C G | −133,800 | −370 | −19,224 |
| 19 mer: G/G | G G A C A T A C C G/G A G T G A A T C G | −133,800 | −369 | −19,472 |
| 19 mer: G/T | G G A C A T A C C G/T A G T G A A T C G | −135,900 | −374 | −19,929 |
| Multiple Internal | | | | |
| Mismatches | | | | |
| A/G; G/G; A/C; T/T | G G A A/G A T A G/G C G A A/C T G A T/T T C G | −89,200 | −256 | −9,933 |
| G/T; C/A; T/C; T/C; G/A | G G G/T C A C/A A C C T/C A G T T/C A A G/A C G | −60,200 | −171 | −7,221 |

Using the calculated ΔH^{o} and ΔS^{o} values and the equation below, the fraction of molecules that are single stranded (random coil) or double stranded at any defined temperature can be calculated. This analysis assumes the transition is at equilibrium and that the equilibrium involves only two states; duplex and random coil. The theoretical melting isotherm based on these calculations is shown below.
ƒ_{random coil}=([Target]−((e^{-(ΔH-TΔS)/RT}([Target]+[Oligo])+1)−((e^{-(ΔH-TΔS)/RT}([Target]+[Oligo])+1)²−4(e^{-(ΔH-TΔS)/RT})²Target][Oligo])^{1/2})/(2e^{-(ΔH-TΔS)/RT}))/[Target]

This analysis allows for an estimation of the fraction of molecules that are in either the duplex or single stranded (random coil) states at any defined temperature. For example, at 37° C., duplex state molecular fraction for the molecules predicted to form a perfect duplex (G) is >99%. Under these same conditions, the duplex state molecular fraction for the molecules predicted to form four or five internal mismatches (4MM and 5MM) is <1%. This supports the contention that homogenous hybridization states can be achieved for sample mixtures where the sequence complexity of the mixture, the length of duplex formed, and ability to choose the target location and hence sequence composition of the duplex allow for the above level of hybridization specificities. Thus, given the encoding design power of the OEHA method, it is possible to create homogeneously encoded target molecular states and thus unambiguously identify an individual target molecule within the sample mixture.

It is also clear from the above example that it is not possible to generate homogenous molecular states for ADOs since their specificity is, by definition, determined by a single base-pair mismatch. In other words, there exits no condition (temperature) where the duplex state molecular fraction for the molecules predicted to form a perfect duplex (G) and that for a single mismatch duplex (either G/A, G/G or G/T) are >99% and <1% respectively. The best differential that can be achieved between the perfect duplex (G) and the single mismatches is at about 56° C. where the duplex molecular fraction is 78% and 14% respectively (FIG. 1; double arrow). Therefore, determining the allele identity of an SNP will require the analysis of a statistically significant number of individual molecules in order to establish the allele identity with a defined degree of certainty.

### SUMMARY

Briefly described, embodiments of this disclosure include polynucleotide analysis systems and method of nanopore analysis. One exemplary polynucleotide analysis system, among others, includes a nanopore analysis system and a first target polynucleotide/allele-discriminating oligonucleotide (ADO) and minor groove binder (MGB) duplex and a second target polynucleotide/ADO-MGB duplex. The nanopore analysis system includes a nanopore device and a nanopore detection system, where the nanopore device includes a structure having a nanopore aperture. The ADO of the first target polynucleotide/ADO-MGB duplex hybridizes to the first allele polynucleotide sequence. The ADO of the second target polynucleotide/ADO-MGB duplex hybridizes to the second allele polynucleotide sequence. The second allele site differs from the first allele site by one nucleotide corresponding to a single nucleotide polymorphism. The nanopore detection system is operative to monitor an electronic signature of the first target polynucleotide/ADO-MGB duplex and the second target polynucleotide/ADO-MGB duplex. The electronic signature for the first target polynucleotide/ADO-MGB duplex and the second target polynucleotide/ADO-MGB duplex are distinguishable.

Methods of nanopore analysis are also provided. One exemplary method, among others, includes: providing a target polynucleotide and an allele-discriminating oligonucleotide (ADO) having a minor groove binder (MGB) on the terminal end of the ADO, wherein the ADO hybridizes to a first allele site of the target polynucleotide and a second allele site of the target polynucleotide, wherein the second allele site differs from the first allele site by one nucleotide corresponding to a single nucleotide polymorphism; forming a first target polynucleotide/ADO-MGB duplex, wherein the ADO hybridizes to the first allele polynucleotide sequence; forming a second target polynucleotide/ADO-MGB duplex, wherein the ADO hybridizes to the second allele polynucleotide sequence; and monitoring an electronic signature of the first target polynucleotide/ADO-MGB duplex and the second target polynucleotide/ADO-MGB duplex, wherein the electronic signature for the first target polynucleotide/ADO-MGB duplex and the second target polynucleotide/ADO-MGB duplex are distinguishable.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

Reference is now made to the following drawings. Note that the components in the drawings are not necessarily to scale.

FIG. 1 is a plot of the theoretical melting isotherm.

FIG. 2 is a schematic of an embodiment of a nanopore analysis system.

FIG. 3 is a flow diagram of a representative process for fabricating a nanopore device.

FIG. 4 is a flow diagram of a representative process describing an aspect of the process described in FIG. 3.

FIG. 5A is a diagram of a representative nanopore device that can be used in the nanopore analysis system of FIG. 2, while FIG. 5B is a representative graph illustrating the measurement of a polynucleotide shown in FIG. 5A.

FIG. 6A is a diagram of a representative nanopore device that can be used in the nanopore analysis system of FIG. 2, while FIG. 6B is a representative graph illustrating the measurement of a polynucleotide shown in FIG. 6A.

FIG. 7A is a diagram of a representative nanopore device that can be used in the nanopore analysis system of FIG. 2.

FIG. 7B is an illustration of a double stranded allele-discriminating oligonucleotide having a pair of minor groove binders used in FIG. 8A.

FIG. 7C is a representative graph illustrating the measurement of a polynucleotide shown in FIG. 7A.

FIG. 8A is a diagram of a representative nanopore device that can be used in the nanopore analysis system of FIG. 2.

FIG. 8B is an illustration of a double stranded allele-discriminating oligonucleotide having a pair of minor groove binders used in FIG. 8A.

FIG. 8C is a representative graph illustrating the measurement of a polynucleotide shown in FIG. 8A.

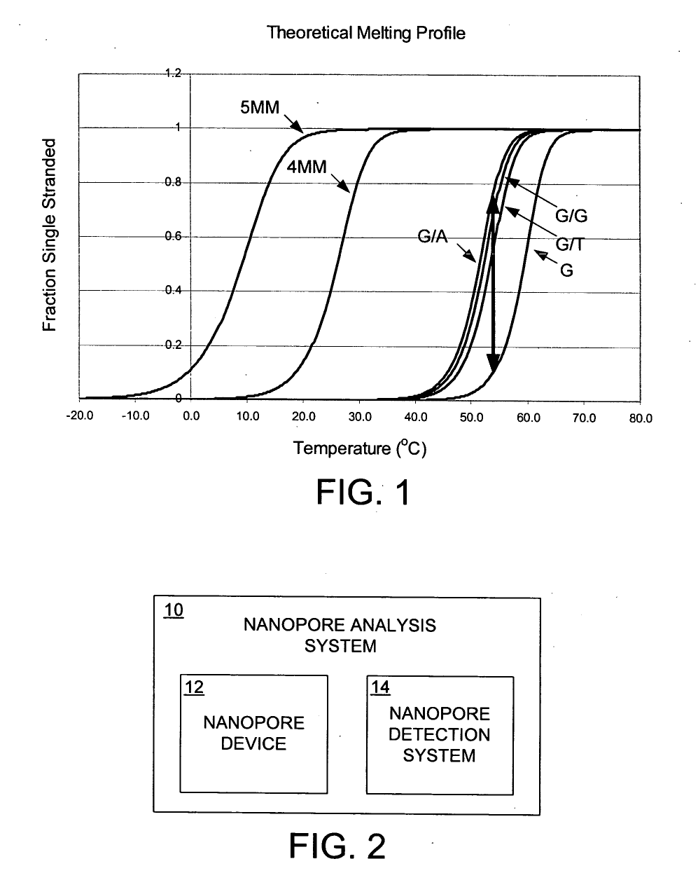

### DETAILED DESCRIPTION

As described in greater detail here, polynucleotide analysis systems and methods of nanopore analysis that can be used for determining polymorphisms are provided. By way of example, some embodiments provide for methods of determining the allele identity of one or more single nucleotide polymorphisms (SNPs) on polynucleotides. In general, target polynucleotides of interest are modified with an allele-discriminating oligonucleotide (ADO) having a minor groove binder (MGB) and then analyzed using a nanopore analysis system. The SNP can be identified using the nanopore analysis system to measure an electronic signature (e.g., ion current and tunneling current) of the modified target polynucleotides. The electronic signature of modified target polynucleotides including the SNP is distinguishable from the electronic signature of modified target polynucleotides not including the SNP. Therefore, the nanopore analysis system can be used to identity the SNP on polynucleotides.

The embodiments of this disclosure addresses, at least in part, the problem of molecular state homogeneity of the ADOs. The embodiments of this disclosure exploit the properties of defined chemical moieties that bind to the minor groove of perfect base-paired duplexes. In the preferred mode, the chemical moieties (MGB) are covalently attached to the ADOs. For example, embodiments of this disclosure are performed by hybridizing the ADOs under conditions that achieve about 100% hybridization to both alleles thereby generating a target molecule having either a perfect base-paired duplex or one with a single base-pair mismatch. The ADO sequence is designed such that the SNP loci are located within the MGB binding domain. When a perfect base-paired duplex is formed between the ADO and the target molecule, the MGB moiety is bound within the minor groove of the duplex. When a single mismatched duplex is formed between the ADO and target molecule, the MGB moiety is excluded from the minor groove. The state of the MGB and hence the allele identity of SNP loci can be determined by the electronic signature as the target molecule traverses a nanopore.

Nanopore sequencing of polynucleotides has been described (U.S. Pat. No. 5,795,782 to Church et al.; U.S. Pat. No. 6,015,714 to Baldarelli et al., the teachings of which are both incorporated herein by reference). In general, nanopore sequencing involves the use of two separate pools of a medium and an interface between the pools. The interface between the pools is capable of interacting sequentially with the individual monomer residues of a polynucleotide present in one of the pools. Interface dependent measurements are continued over time, as individual monomer residues of the polynucleotide interact sequentially with the interface, yielding data suitable to infer a monomer-dependent characteristic of the polynucleotide. The monomer-dependent characterization achieved by nanopore sequencing may include identifying physical characteristics such as, but not limited to, the number and composition of monomers that make up each individual polynucleotide, in sequential order.

The term “sequencing” as used herein means determining the sequential order of nucleotides in a polynucleotide molecule. Sequencing as used herein includes in the scope of its definition, determining the presence of single nucleotide polymorphisms (SNPs). In addition, sequencing can include determining the nucleotide sequence of a polynucleotide in which the sequence or portions thereof was previously unknown or known.

FIG. 2 illustrates a representative embodiment of a nanopore analysis system 10 that can be used in nanopore sequencing. The nanopore analysis system 10 includes, but is not limited to, a nanopore device 12 and a nanopore detection system 14. The nanopore device 12 and the nanopore detection system 14 are communicatively coupled so that data regarding a polynucleotide can be measured.

The nanopore detection system 14 includes, but is not limited to, electronic equipment capable of measuring electronic characteristics of the polynucleotide as it interacts with a nanopore aperture in a structure of the nanopore detection system, a computer system capable of controlling the electronic measurement of the characteristics and storing the corresponding data, control equipment capable of controlling the conditions of the nanopore device, and components that are included in the nanopore device that are used to perform the electronic measurements.

The nanopore detection system 14 can measure electronic characteristics such as, but not limited to, the amplitude or duration of individual conductance or electron tunneling current changes across a nanopore aperture. Such changes can identify the monomers in sequence, as each monomer has a characteristic conductance change signature. For instance, the volume, shape, or charges on each monomer can affect conductance in a characteristic way. Therefore, polynucleotides may produce distinguishable electronic signatures based on volume and/or shape changes.

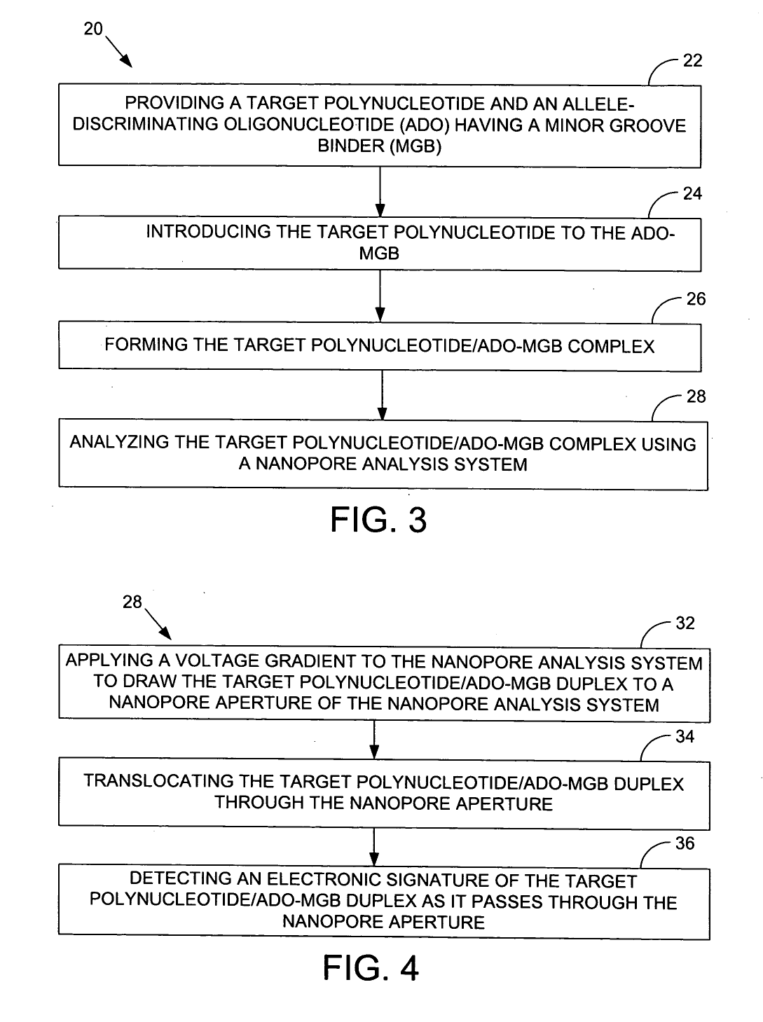

FIG. 3 is a flow diagram illustrating a representative process 20 for using the nanopore analysis system 10. As shown in FIG. 3, the functionality (or method) may be construed as beginning at block 22, where at least one target polynucleotide (e.g., a chromosome fragment) and an allele-discriminating oligonucleotide (ADO) having a minor groove binder (MGB), are provided. The target polynucleotides can include either a first polynucleotide sequence including the first allele or a second polynucleotide sequence including a second allele. An “allele site” refers to a defined polynucleotide sequence within the target polynucleotide that includes a sequence difference (i.e., usually a single nucleotide difference). Preferably, the single nucleotide difference in the target polynucleotide is located near the 3′ end of the allele site, which places it within the last five 5′-terminal nucleotides of the complementary ADO. The first allele site can include about 6 to 40 nucleotides, about 15 and 30 nucleotides, and about 20 and 25 nucleotides. The exact length of the allele site and hence length of the complementary ADO is determined, at least in part, by the sequence complexity of the target mixture. The length of the ADO is sufficient to ensure specific hybridization to the desired allele site within the target mixture. It should be noted that the target polynucldotide could include zero or one or more allele sites, where one or more ADO-MGBs can be used to identify the allele sites.

The ADO includes a nucleotide sequence that substantially hybridizes to the first allele site and the second allele site of the target polynucleotide and preferably to the exclusion of other sequences within the target mixture. In addition, the ADO includes an MGB positioned such that it can bind into the minor groove of the duplex formed between the target allele site and the ADO. Preferably, the MGB is positioned such that it binds into the region of the duplex minor groove including the site of the single nucleotide polymorphism within the target allele site.

The MGB can include, but is not limited to, antitumor antibiotics (e.g., CC-1065, durocarmycin A, and duocarmycin SA), Netropis, and distamycin, which bind to A/T rich minor grooves (e.g., U.S. Pat. No. 6,312,894). In addition, the MGB can include, but is not limited to, a class of polypyrroles derived from the conjugation of N-methylpyrrole carboxamide and N-3-carbamoyl-1,2-dihydro-3H-pyrrolo[3,2-e]indole-7-carboxylate, which bind to all four base-pairs. The binding of these MGB moieties to the minor groove is achieved primarily through van der Waals' and various hydrogen bonding interactions (e.g., Uytterhoeven et al., Eur. J. Biochem, 269: 2868-2877 (2002)).

In block 24, the target polynucleotide is introduced to the ADO-MGB. The target polynucleotide and the ADO-MGB are disposed on the same side (cis or trans) of the nanopore device 12 and allowed to interact. Alternatively, the target polynucleotide and the ADO-MGB are mixed prior to introduction to the nanopore device 12. In block 26, a target polynucleotide ADO-MGB duplex is formed.

The target polynucleotide ADO-MGB duplex can include a first target polynucleotide/ADO-MGB duplex and a second target polynucleotide/ADO-MGB duplex. In the first target polynucleotide/ADO-MGB duplex, the ADO hybridizes to the first allele site of the first target polynucleotide. The hybridization of the ADO with the first allele site forms a perfect duplex. It should be noted from above that the MGB's are known to bind to the minor groove of double stranded duplexes. As a result, the MGB substantially fits within the minor groove of the duplex and is substantially indistinguishable from a duplex including the MGB using nanopore analysis.

In the second target polynucleotide/ADO-MGB duplex, the ADO hybridizes to the second allele site of the second target polynucleotide. However, the hybridization does not form a prefect duplex because of a mismatch between the ADO and the second allele site due to the SNP at the terminal end of the second allele polynucleotide sequence. As a result, the MGB does not substantially fit within the minor groove of the duplex. Thus, the second target polynucleotide/ADO-MGB duplex is distinguishable from the first target polynucleotide/ADO-MGB duplex using nanopore analysis because of, at least, the duplex mismatch and the MGB.

Subsequently, in block 28, the target polynucleotide ADO-MGB duplex is analyzed using the nanopore analysis system 10. In general, an electronic signature corresponding to the target polynucleotide ADO-MGB duplex can be obtained using the nanopore analysis system 10. In particular, an electronic signature corresponding to the first target polynucleotide/ADO-MGB duplex and an electronic signature corresponding to the second target polynucleotide/ADO-MGB duplex can be measured. As discussed above, the electronic signature corresponding to the first target polynucleotide/ADO-MGB duplex and the electronic signature corresponding to the second target polynucleotide/ADO-MGB duplex are distinguishable. Therefore, a determination can be made as to whether the target polynucleotides include the first allele site or the second allele polynucleotide sequence. If the target polynucleotide includes the second allele polynucleotide sequence, then the target polynucleotide includes the SNP.

FIG. 4 is a flow diagram illustrating a representative embodiment of the analysis of the target polynucleotide ADO-MGB duplex shown in block 28 of FIG. 3. In block 32, a voltage gradient is applied to the nanopore device 12 to draw the target polynucleotide ADO-MGB duplex to the cis side of the nanopore aperture 44. In block 34, the target polynucleotide ADO-MGB duplex is translocated through the nanopore aperture 44. In block 36, the electronic signature of the target polynucleotide ADO-MGB duplex is measured as the target polynucleotide ADO-MGB duplex translocates through the nanopore aperture. Additional details regarding the nanopore analysis system 10 are described below.

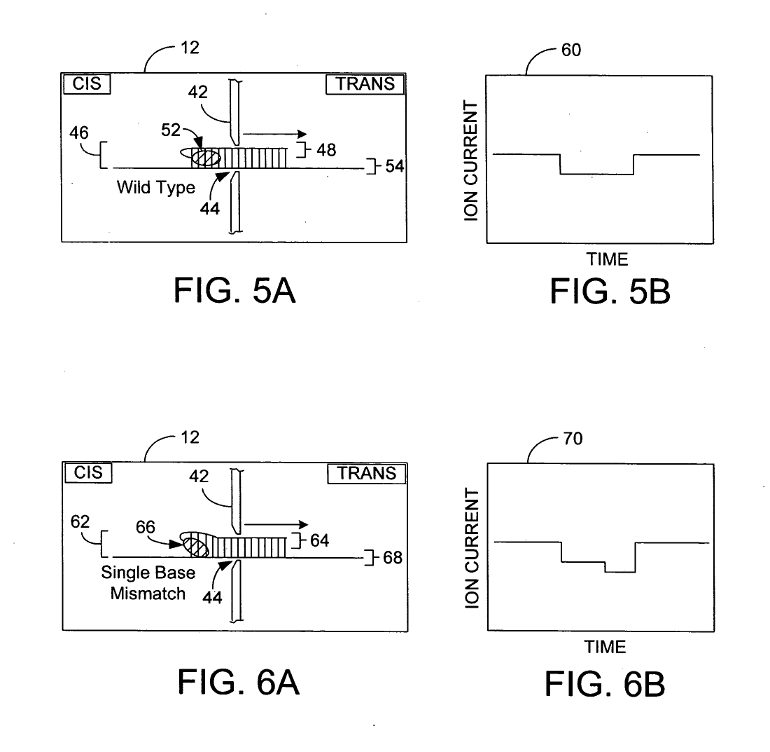

FIG. 5A illustrates a representative embodiment of the nanopore device 12. The nanopore device 12 includes, but is not limited to, a structure 42 that separates two independent adjacent pools of a medium. The two adjacent pools are located on the cis side and the trans side of the nanopore device 12. The structure 42 includes, but is not limited to, at least one nanopore aperture 44 so dimensioned as to allow sequential monomer-by-monomer translocation (i.e., passage) from one pool to another of only one polynucleotide at a time, and detection components that can be used to perform measurements of the target polynucleotide.

Exemplary detection components have been described in WO 00/79257 and can include, but are not limited to, electrodes directly associated with the structure 42 at or near the nanopore aperture 44, and electrodes placed within the cis and trans pools. The electrodes may be capable of, but not limited to, detecting electronic differences across the two pools or electron tunneling currents across the nanopore aperture 44.

A target polynucleotide 54 and an ADO-MGB 48 are introduced to one another and form a target polynucleotide ADO-MGB duplex 54. The ADO hybridizes to a first allele site (wild type) of the target polynucleotide 54 to form a perfect duplex between the ADO and the first allele polynucleotide sequence. Since a perfect duplex is formed, the MGB 52 binds in the minor groove of the duplex.

FIG. SA illustrates the target polynucleotide ADO-MGB duplex 46 translocating through the nanopore aperture 44. As the target polynucleotide ADO-MGB duplex 46 translocates through the nanopore aperture 44, electronic measurements as a function of time are taken by the nanopore detection system 14 (FIG. 5B). The electronic measurements can be used to identify the target polynucleotide ADO-MGB duplex 46 and distinguish it from other sequences (e.g., target polynucleotide ADO-MGB duplex 62 in FIG. 5A as shown in FIGS. 5B and 6B).

The structure 42 can be made of materials such as, but not limited to, silicon nitride, silicon oxide, mica, and polyimide. The structure 42 can include, but is not limited to, detection electrodes and detection integrated circuitry. The structure 42 includes one nanopore aperture 44 but could include two or more nanopore apertures. The nanopore aperture 44 is dimensioned so that the target polynucleotide ADO-MGB duplex 46 can translocate through the nanopore aperture 44. The nanopore aperture 44 can have a diameter of about 3 to 5 nanometers.

The medium disposed in the pools on either side of the substrate 42 may be any fluid that permits adequate polynucleotide mobility for substrate interaction. Typically, the medium is a liquid, usually aqueous solutions or other liquids or solutions in which the polynucleotides can be distributed. When an electrically conductive medium is used, it can be any medium which is able to carry electrical current. Such solutions generally contain ions as the current-conducting agents (e.g., sodium, potassium, chloride, calcium, cesium, barium, sulfate, or phosphate).

Conductance across the nanopore aperture 44 can be determined by measuring the flow of current across the nanopore aperture via the conducting medium. A voltage difference can be imposed across the barrier between the pools using appropriate electronic equipment. Alternatively, an electrochemical gradient may be established by a difference in the ionic composition of the two pools of medium, either with different ions in each pool, or different concentrations of at least one of the ions in the solutions or media of the pools. Conductance changes are measured by the nanopore detection system 14 and are indicative of monomer, volume, and/or shape characteristics.

The target polynucleotide ADO-MGB duplex 46 may remain in its original pool (not depicted), or it may translocate through the nanopore aperture 44 into the other pool. In either situation, the target polynucleotide ADO-MGB duplex 46 moves in relation to the nanopore aperture 44, individual nucleotides interact sequentially with the nanopore aperture 44 to induce a change in the conductance of the nanopore aperture 44. In embodiments where the target polynucleotide ADO-MGB duplex 46 traverses across the nanopore aperture 44 without crossing into the other pool, the target polynucleotide ADO-MGB duplex 46 is close enough to the nanopore aperture 24 for its nucleotides to interact with the nanopore aperture 44 passage and bring about the conductance changes, which are indicative of the target polynucleotide ADO-MGB duplex 46 characteristics.

FIG. 6A illustrates a representative embodiment of the nanopore device 12. The nanopore device 12 includes, but is not limited to, a structure 42 that separates two independent adjacent pools of a medium. The two adjacent pools are located on the cis side and the trans side of the nanopore device 12. The structure 42 includes, but is not limited to, at least one nanopore aperture 44 so dimensioned as to allow sequential monomer-by-monomer translocation (i.e., passage) from one pool to another of only one polynucleotide at a time, and detection components that can be used to perform electronic measurements of the target polynucleotide.

A target polynucleotide 68 and an ADO-MGB 64 are introduced to one another and form a target polynucleotide ADO-MGB duplex 62. The ADO hybridizes to a second allele site of the target polynucleotide 68 but does not form a perfect duplex between the ADO and the second allele polynucleotide sequence. The second allele site and the first allele site include at least one difference in the nucleotide sequence. The difference is located in the last five terminal nucleotides of the sequence and the difference in nucleotide sequence corresponds to an SNP. Since a non-perfect duplex is formed and the mismatch occurs in the terminal five nucleotides, the MGB 66 does not bind substantially in the minor groove of the duplex.

FIG. 6A illustrates the target polynucleotide ADO-MGB duplex 62 translocating through the nanopore aperture 44. As the target polynucleotide ADO-MGB duplex 62 translocates through the nanopore aperture 44, electronic measurements as a function of time are taken by the nanopore detection system 14 (FIG. 6B). The electronic measurements can be used to identify the target polynucleotide ADO-MGB duplex 62 and distinguish it from the target polynucleotide ADO-MGB duplex 46 in FIGS. 5A and 5B.

FIGS. 5B and 6B illustrate graphs 60 and 70 of electronic measurements as a function of time for the target polynucleotide ADO-MGB duplexes 46 and 62, respectively. As a result of the mismatch of the ADO/second allele site of the target polynucleotide 68, the electronic graph 70 of the target polynucleotide ADO-MGB duplex 62 is distinguishable from the electronic graph 60 of the target polynucleotide ADO-MGB duplex 46. Therefore, nanopore analysis systems 10 incorporating the ADO-MGB to form a duplex with the target polynucleotide can be used to identify SNP's.

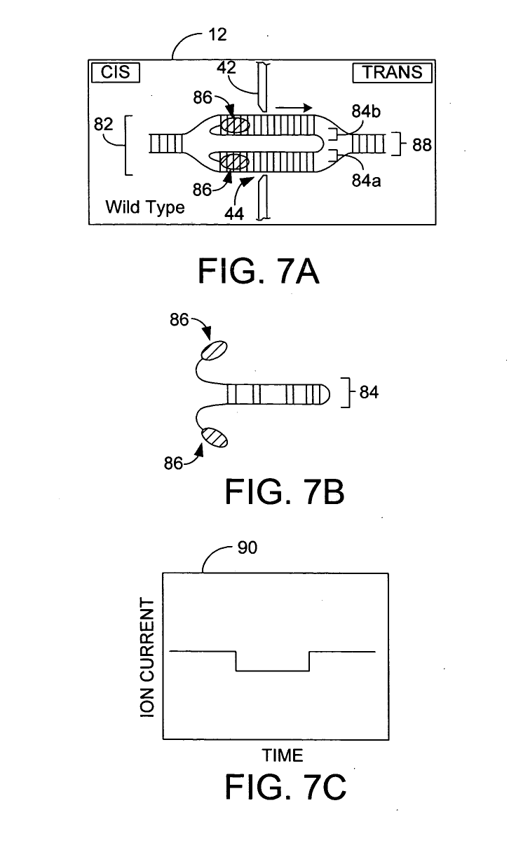

FIG. 7A illustrates a representative embodiment of the nanopore device 12. The nanopore device 12 includes, but is not limited to, a structure 42 that separates two independent adjacent pools of a medium. The two adjacent pools are located on the cis side and the trans side of the nanopore device 12. The structure 42 includes, but is not limited to, at least one nanopore aperture 44 so dimensioned as to allow sequential monomer-by-monomer translocation (i.e., passage) from one pool to another of only one polynucleotide at a time, and detection components that can be used to perform measurements of the target polynucleotide.

A double-stranded target polynucleotide 88 and an MGB-ADO-MGB 88 (FIG. 7B) are introduced to one another. The ADO includes a sequence complementary to both strands of the duplex target polynucleotide 88 separated by a short (4 nucleotide) linker region having an MGB 86 attached to both the 3′ and 5′ termini of the ADO. In an embodiment, the ADO comprises modified nucleotides that do not form stable base-pairs with their complementary partner in the ADO strand but can form stable base-pairs with their complementary partner in the target strand (see for example: US20030211474, US20030104428, EP1072679). The use of these types of unstructured nucleic acids (UNAs) will prevent the ADO from forming a stable hairpin structure thereby facilitating the strand invasion of the double-stranded target polynucleotide 88 by the MGB-ADO-MGB molecule 84. The MGB-ADO-MGB 84 hybridizes to the first allele site (wild type) of the double-stranded target polynucleotide 88 to form a double-duplex between the MGB-ADO-MGB 84a and 84b and the first allele polynucleotide sequence. Since perfect duplexes are formed between the MGB-ADO-MGB 84a and 84b and the two target polynucleotide strands 88, the MGB 86 moieties bind into the minor groove of the duplexes.

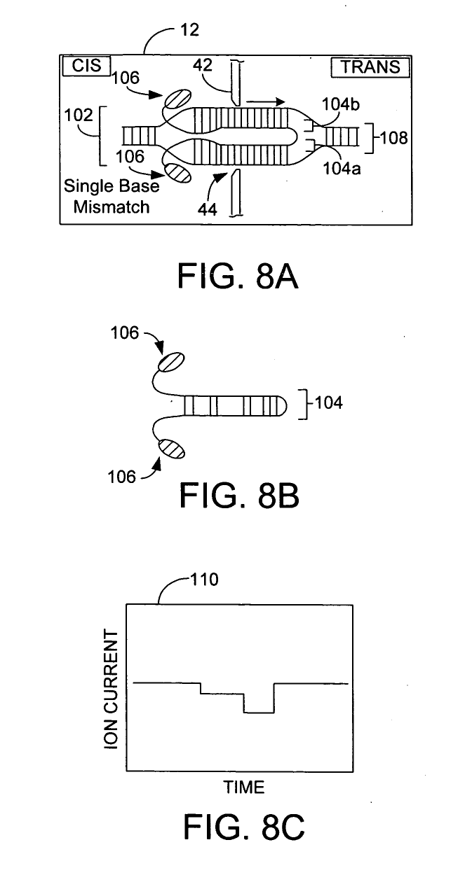

FIG. 7A illustrates the double-stranded target polynucleotide MGB-ADO-MGB double-duplex 82 translocating through the nanopore aperture 44. As the double-stranded target polynucleotide MGB-ADO-MGB double-duplex 82 translocates through the nanopore aperture 44, electronic measurements as a function of time are taken by the nanopore detection system 14 (FIG. 2). The electronic measurements can be used to identify the double-stranded target polynucleotide MGB-ADO-MGB duplex 82 and distinguish it from other sequences (e.g., double-stranded target polynucleotide MGB-ADO-MGB duplex 102 in FIG. 8A as shown in FIGS. 7C and 8C upon comparison thereof).

The structure 42 can be made of materials such as, but not limited to, silicon nitride, silicon oxide, mica, and polyimide. The structure 42 can include, but is not limited to, detection electrodes and detection integrated circuitry. The structure 42 includes one nanopore aperture 44 but could include two or more nanopore apertures. The nanopore aperture 44 is dimensioned so that the double-stranded target polynucleotide including the MGB-ADO-MGB double-duplex 82 can translocate through the nanopore aperture 44. The nanopore aperture 44 can have a diameter of about 5 to 7 nanometers.

Conductance across the nanopore aperture 44 can be determined by measuring the flow of current across the nanopore aperture 44 via the conducting medium. The medium disposed in the pools on either side of the substrate 42 may be any fluid that permits adequate polynucleotide mobility for substrate interaction as described above. A voltage difference can be imposed across the barrier between the pools using appropriate electronic equipment. Alternatively, an electrochemical gradient may be established by a difference in the ionic composition of the two pools of medium, either with different ions in each pool, or different concentrations of at least one of the ions in the solutions or media of the pools. Conductance changes are measured by the nanopore detection system 14 and are indicative of monomer, volume, and/or shape characteristics.

The double-stranded target polynucleotide MGB-ADO-MGB double-duplex 82 may remain in its original pool (not depicted), or it may translocate through the nanopore aperture 44 into the other pool. In either situation, the double-stranded target polynucleotide MGB-ADO-MGB double-duplex 82 moves in relation to the nanopore aperture 44, individual nucleotides interact sequentially with the nanopore aperture 44 to induce a change in the conductance of the nanopore aperture 44. In embodiments where the double-stranded target polynucleotide MGB-ADO-MGB double-duplex 82 traverses across the nanopore aperture 44 without crossing into the other pool, the double-stranded target polynucleotide MGB-ADO-MGB double-duplex 82 is close enough to the nanopore aperture 44 for its nucleotides to interact with the nanopore aperture 44 passage and bring about the conductance changes, which are indicative of the double-stranded target polynucleotide MGB-ADO-MGB double-duplex 82 characteristics.

FIG. 8A illustrates a representative embodiment of the nanopore device 12. The nanopore device 12 includes, but is not limited to, a structure 44 as described above in reference to FIG. 7A. A double stranded target polynucleotide 108 and an MBG-ADO-MGB 104 are introduced to one another. The ADO strand invades at a second allele site of the duplex target polynucleotide 108 but does not form a perfect double-duplex between the ADO sequences and their complements within the second allele polynucleotide sequence. The second allele site and the first allele site include at least one difference in the nucleotide sequence. The difference is located in the last five terminal nucleotides of the sequence and the difference in nucleotide sequence corresponds to an SNP. Since non-perfect duplexes are formed between the ADO and the mismatch occurs in the terminal five nucleotides, the MGB 106 does not bind substantially in the minor groove of the duplex.

FIG. 8A illustrates the double-stranded target polynucleotide MGB-ADO-MGB double-duplex 102 translocating through the nanopore aperture 44. As the double-stranded target polynucleotide MGB-ADO-MGB double-duplex 102 translocates through the nanopore aperture 44, electronic measurements as a function of time, are taken by the nanopore detection system 14 (FIG. 2). The electronic measurements can be used to identify the double-stranded target polynucleotide MGB-ADO-MGB double-duplex 102 and distinguish it from the double-stranded target polynucleotide MGB-ADO-MGB double-duplex 82 in FIGS. 7A and 7C.

FIGS. 7C and 8C illustrate graphs 90 and 110 of electronic measurements as a function of time for the double-stranded target polynucleotide ADO-MGB duplexes 82 and 102, respectively. As a result of the mismatch of the ADO/second allele site of the target polynucleotide 108, the electronic graph 110 of the double-stranded target polynucleotide ADO-MGB duplex 102 is distinguishable from the electronic graph 90 of the double-stranded target polynucleotide ADO-MGB duplex 82. Therefore, nanopore analysis systems 10 incorporating the ADO-MGB to form a duplex with the target polynucleotide can be used to identify SNP's.

It should be emphasized that many variations and modifications may be made to the above-described embodiments. All such modifications and variations are intended to be included herein within the scope of this disclosure and protected by the following claims.

We claim at least the following:

1. A method of nanopore analysis, comprising:
providing a target polynucleotide and an allele-discriminating oligonucleotide (ADO) having a first minor groove binder (MGB) on a terminal end of the ADO, wherein the ADO hybridizes to an allele site selected from a first allele site of the target polynucleotide and a second allele site of the target polynucleotide, wherein the second allele site differs from the first allele site by one nucleotide corresponding to a single nucleotide polymorphism;
forming at least one duplex selected from:
a first target polynucleotide/ADO-MGB duplex, wherein the ADO hybridizes to the first allele site of the target polynucleotide; and
a second target polynucleotide/ADO-MGB duplex, wherein the ADO hybridizes to the second allele site of the target polynucleotide; and
monitoring an electronic signature of the duplex, wherein the electronic signature for the first target polynucleotide/ADO-MGB duplex and the second target polynucleotide/ADO-MGB duplex are distinguishable.

2. The method of claim 1, wherein the single nucleotide polymorphism is positioned in at least one of the last five terminal nucleotides of the second allele site of the target polynucleotide.

3. The method of claim 1, wherein the first MGB is positioned substantially within a minor groove of the first target polynucleotide/ADO-MGB duplex, and wherein the first MGB is positioned substantially out of a minor groove of the second target polynucleotide/ADO-MGB duplex.

4. The method of claim 1, wherein monitoring comprises:
detecting the electronic signature using a nanopore analysis system.

5. The method of claim 4, further comprising:
applying a voltage gradient to the nanopore analysis system to draw the first target polynucleotide/ADO-MGB duplex and the second target polynucleotide/ADO-MGB duplex to a nanopore aperture of the nanopore analysis system; and
translocating the first target polynucleotide/ADO-MGB duplex and the second target polynucleotide/ADO-MGB duplex through the nanopore aperture.

6. The method of claim 1, wherein the first MGB is selected from CC-1065, durocarmycin A, duocarmycin SA, Netropis, distamycin, and a class of polypyrroles derived from the conjugation of N-methylpyrrole carboxamide and N-3-carbamoyl-1,2-dihydro-3H-pyrrolo[3,2-e]indole-7-carboxylate.

7. A method of nanopore analysis, comprising:
providing a nanopore analysis system;
providing a target polynucleotide and an allele-discriminating oligonucleotide (ADO) having a first minor groove binder (MGB) on a terminal end of the ADO, wherein the ADO hybridizes to an allele site selected from a first allele site of the target polynucleotide and a second allele site of the target polynucleotide, wherein the second allele site differs from the first allele site by one nucleotide that includes a single nucleotide polymorphism, wherein the single nucleotide polymorphism is positioned in at least one of the last five terminal nucleotides of the second allele site;
exposing the target polynucleotide to the ADO having the first MGB to form a duplex selected from a first target polynucleotide/ADO-MGB duplex and a second target polynucleotide/ADO-MGB duplex, wherein the first MGB is positioned substantially within a minor groove of the first target polynucleotide/ADO-MGB duplex when the ADO hybridizes to the first allele site of the target polynucleotide, and wherein the first MGB is positioned substantially out of a minor groove of the second target polynucleotide/ADO-MGB duplex when the ADO hybridizes to the second allele site of the target polynucleotide; and
determining the presence of the first MGB relative the minor groove of one of the first target polynucleotide/ADO-MGB duplex and the second target polynucleotide/ADO-MGB duplex using the nanopore analysis system, and wherein the presence of the first MGB in the minor groove of the first target polynucleotide/ADO-MGB duplex indicates that the target polynucleotide does not include the single nucleotide polymorphism.

8. The method of claim 7, further comprising:
applying a voltage gradient to the nanopore analysis system to draw one of the first target polynucleotide/ADO-MGB duplex and the second target polynucleotide/ADO-MGB duplex to a nanopore aperture of the nanopore analysis system;
translocating one of the first target polynucleotide/ADO-MGB duplex and the second target polynucleotide/ADO-MGB duplex through the nanopore aperture; and
detecting an electronic signature of one of the first target polynucleotide/ADO-MGB duplex the second target polynucleotide/ADO-MGB duplex as it passes through the nanopore aperture, wherein the electronic signature for the first target polynucleotide/ADO-MGB duplex is distinguishable from the second target polynucleotide/ADO-MGB duplex.

9. The method of claim 7, wherein the target polynucleotide is a single strand polynucleotide and the ADO is a single strand ADO polynucleotide having one first MGB on the terminal end of the ADO.

10. The method of claim 7, wherein the ADO further comprises a second MGB on a second terminal end of the ADO, wherein the ADO hybridizes to a third allele site of the target polynucleotide and a fourth allele site of the target polynucleotide, wherein the fourth allele site differs from the third allele site by one nucleotide that includes a second single nucleotide polymorphism, wherein the single nucleotide polymorphism is positioned in at least one of the last five terminal nucleotides of the fourth allele site of the target polynucleotide, and further comprising:
exposing the target polynucleotide to the ADO having the second MGB to form one of a third target polynucleotide/ADO-MGB duplex and a fourth target polynucleotide/ADO-MGB duplex, wherein the second MGB is positioned substantially within a minor groove of the third target polynucleotide/ADO-MGB duplex when the ADO hybridizes to the third allele site of the target polynucleotide, and wherein the second MGB is positioned substantially out of a minor groove of the fourth target polynucleotide/ADO-MGB duplex when the ADO hybridizes to the fourth allele site of the target polynucleotide; and
determining the presence of the second MGB relative to the minor groove of one of the third target polynucleotide/ADO-MGB duplex and the fourth target polynucleotide/ADO-MGB duplex using the nanopore analysis system, wherein the presence of the second MGB in the minor groove of the third target polynucleotide/ADO-MGB duplex indicates that the target polynucleotide does not include the single nucleotide polymorphism.

11. The method of claim 7, wherein the MGB is selected from CC-1065, durocarmycin A, duocarmycin SA, Netropis, distamycin, and a class of polypyrroles derived from the conjugation of N-methylpyrrole carboxamide and N-3-carbamoyl-1,2-dihydro-3H-pyrrolo[3,2-e]indole-7-carboxylate.

12. A polynucleotide analysis system, comprising:
a nanopore analysis system including a nanopore device and a nanopore detection system, wherein the nanopore device includes a structure having a nanopore aperture; and
a duplex selected from a first target polynucleotide/allele-discriminating oligonucleotide (ADO) and minor groove binder (MGB) duplex and a second target polynucleotide/ADO-MGB duplex, wherein the ADO of the first target polynucleotide/ADO-MGB duplex hybridizes to a first allele of the target polynucleotide, wherein the ADO of the second target polynucleotide/ADO-MGB duplex hybridizes to a second allele of the target polynucleotide, wherein the second allele site differs from the first allele site by one nucleotide corresponding to a single nucleotide polymorphism, wherein the nanopore detection system is operative to distinguish an electronic signature of the first target polynucleotide/ADO-MGB duplex and the second target polynucleotide/ADO-MGB duplex, and wherein the electronic signature for the first target polynucleotide/ADO-MGB duplex and the second target polynucleotide/ADO-MGB duplex are distinguishable.

13. The polynucleotide analysis system of claim 12, wherein the nanopore detection system is operative to detect an electrical characteristic of the first target polynucleotide/ADO-MGB duplex and the second target polynucleotide/ADO-MGB duplex translocating the nanopore aperture.

14. The polynucleotide analysis system of claim 12, wherein the electrical signature includes the presence of the first MGB substantially in the minor groove of the first target polynucleotide/ADO-MGB duplex.

15. The polynucleotide analysis system of claim 13, wherein the electrical signature includes the absence of the first MGB substantially in the minor groove of the second target polynucleotide/ADO-MGB duplex.

16. The polynucleotide analysis system of claim 12, further comprising a means for detecting an electrical signature of the first target polynucleotide/ADO-MGB duplex and the second target polynucleotide/ADO-MGB duplex translocating the nanopore aperture.
